(19) United States
(12) Patent Application Publication
(10) Pub. No.: US 2005/0186260 A1
(43) Pub. Date: Aug. 25, 2005

(54) Medicated gel foam and method of use

(76) Inventors: Philip Paul Narini, Oshawa (CA); Brenda Matloub, Waukesha, WI (US); Haitham Matloub, Waukesha, WI (US)

Correspondence Address: LADAS & PARRY LLP, 224 SOUTH MICHIGAN AVENUE, SUITE 1600, CHICAGO, IL, 60604, US

(21) Appl. No.: 10/782,553

(22) Filed: Feb. 19, 2004

(51) Int. Cl. A61L 15/16; A61K 38/18

(52) U.S. Cl. 424/445; 514/12

(57) ABSTRACT

A method and improved foam structure for use in vacuum assisted closure therapy comprising an open cell foam structure coated with a therapeutic agent.

### FIELD OF THE INVENTION

This invention relates to an improved foam structure or pad that is coated with a therapeutic agent and, more specifically, to an improved open cell medicated foam pad and its method of use in treating wounds.

### BACKGROUND OF THE INVENTION

Promoting the debridement and healing of the wound by negative pressure or sub-atmospheric pressure for an extended period of time was described by Fleischmann et al. in 1993. Fleischmann, W Strecker W, Bombelli M, Kinzl L. [Vacuum sealing as treatment of soft tissue damage in open fractures]. Unfallchirug 1993; 96(9): 488-92. The report described the successful use of the sub-atmospheric pressure technique in 15 patients with open bone fractures. They reported that the negative pressure therapy treatment resulted in efficient healing and conditioning of the wound with removal of grandular tissue. In subsequent publications Fleischmann et al. described the treatment of 25 patients with problems of a lower limb and 313 patients with acute and chronic infections of various types, Fleischmann W, Lang E, Kinzl L. [Vacuum assisted wound closure after dermatofasciotomy of the lower extremity]. Unfallchirug 1996; 99(4): 283-7; Fleischmann W, Lang E, Russ M. [Treatment of infection by vacuum sealing]. Unfallchirug 1997; 100(4): 301-4, the average duration of the vacuum therapy treatment for patients with problems of the lower limb was 12.7 days with 2.1 dressing changes per patient. The wounds were subsequently either closed by secondary suturing or by skin graphs following partial closure by suturing.

Negative pressure wound therapy assists in wound closure by applying localized negative (sub-atmospheric) pressure to help promote wound healing. Vacuum pressure is applied to a special dressing positioned in the wound cavity or over a flap or graft. This pressure-distributing wound packing helps remove fluids from the wound and promote the normal healing process.

The negative pressure therapy uses open-cell reticulated foam that can be cut to the shape of the wound, or can be placed side by side or layered to treat very large wounds. A tube in contact with the foam allows the application of vacuum pressure for the removal of excess wound fluid. The dressing and distal evacuation tube are covered by a transparent, occlusive drape that provides a seal which allows the application of vacuum pressure to the system.

The free end of the evacuation tube is attached to a canister reservoir, which fits into a microprocessor-controlled vacuum unit and collects the fluids drawn away from the wound. The vacuum unit provides continuous or intermittent negative pressure selected to meet the needs of the wound being treated. The pressure can be adjusted within a range that has been demonstrated to provide optimal fluid removal without placing the delicate wound tissue at risk of injury.

The application of negative pressure therapy to a wound provides a moist wound-healing environment. A moist wound-healing environment is the standard of care for wound healing. Removal of excess interstitial fluid also can lead to removal of excess proteinases present in the periwound environment. Metalloproteinases are known to bind and degrade growth factors before the growth factor can reach its target tissue. With inhibitors removed, growth factors can stimulate cell proliferation and migration. Removal of excess interstitial fluid can naturally help decrease periwound induration (swelling) further helping to promote wound healing.

The evacuation of air from the open cells of the foam causes the foam to collapse on itself and provide a mechanical distraction, or stretching, of the soft tissues. This mechanical force helps draw the wound edges towards the center of the wound, hence, assisting in promotion of wound closure.

In 1995, a commercial system for promoting vacuum assisted closure was introduced into the United States. The equipment is entitled the V.A.C. for vacuum assistance closure, manufactured by Kinetics Concepts, Inc., San Antonio, Tex.

Negative pressure therapy offers many benefits of high technology. In the V.A.C system the standard dressing change routine of every 48 hours (12 hours for infected wounds) can result in less disturbance to the wound and improved patient comfort.

U.S. Pat. No. 6,135,116 describes a method and apparatus for providing concurrent applications of intermittent pneumatic compression therapy and vacuum assisted closure therapy mainly to a patient's foot or toe. The therapy generally comprises a wound dressing for introduction of negative pressure into a wound on a patient's foot and a foot wrap for application of positive, compressive forces to substantially all of the patients foot. A suction pump, having a vacuum sensor and first feedback mechanism, supplies negative pressure to the wound dressing. A pressurized gas, having an associated pressure transducer and second feedback mechanism, supplies positive force to the foot wrap.

U.S. Pat. No. 6,458,109 describes a wound treatment apparatus with a bandage system configured to control the environment adjacent to the wound. The apparatus includes a bandage configured to cover a wound and provide a seal about the perimeter of the wound and cavity over the wound. A fluid supply conduit and a fluid drainage conduit are each in communication with the cavity. A nebulizer is coupled to the supply conduit to supply medicinal fluid to the wound.

In the foregoing references, describing the vacuum assisted closing system, the method of treatment is substantially similar to negative pressure wound therapy. A bandage or dressing is used on the open wound. Typically, a piece of foam with an open cell structure is placed onto the wound and a drain with lateral perforations is laid on top of it. The open cell structure and drain are then covered with a membrane, preferably an adhesive transparent membrane secured to the skin around the margin of the wound. The distal end of the drain tube is connected to a vacuum source so that fluid is drawn from the wound through the foam into a reservoir for disposal. The membrane allows for a partial vacuum to form within the wound and prevents the ingress of air. Vacuuming of the wound reduces its volume and facilitates the removal of fluids. The open cell foam covers the entire surface area of the wound, and uniformly exposes the wound to the negative pressure of the vacuum. The open cell foam prevents occlusion of the perforations in the drain in contact with the base or edges of the wound and helps prevent tissue necrosis.

Problems associated with applying a negative pressure to a wound include: 1. Tissue growth into the foam, 2. Potential damage of delicate structures such as blood vessels and internal organs (i.e. bowel), 3. Adhesion of the foam to the wound base causing repeated trauma (therefore increasing pain and increased healing time) with dressing changes. Also, an improved understanding of factors that modulate wound healing has led to the use of natural and synthetic substances to promote wound healing. There is no current mechanism to add these substances to the foam used with vacuum assisted closure. Hence, there is a need for an improved or modified foam that is treated with one or more therapeutic agents, that is, pharmaceutical or natural compositions, to aid in the treatment of the wound. Possible benefits are inhibiting the growth of bacteria, promoting healing of the wound, and prevention of adhesion of the foam to the wound, which will improve patient comfort.

### SUMMARY OF THE INVENTION

The present invention generally comprises a wound dressing or a medicated foam structure comprising a foam structure having at least first and second sides and a therapeutic agent applied on at least one or both of the sides. The medicated foam structure may be used individually or preferably in a V.A.C. system or in negative pressure therapy for treating a wound. The medicated foam structure comprises porous open cells with each cell having a diameter of about 500 to about 700 microns to hold and release one or more therapeutic agents.

Therefore, it is an object of the present invention to provide a medicated foam structure for cleansing or debridement of wounds.

It is another object of the present invention to provide a medicated foam pad to kill or inhibit the growth of bacteria near or within the wound.

It is another object of the present invention to provide a medicated foam pad to be used with negative pressure therapy on a wound.

It is another object of the present invention to provide a foam structure to be used with negative pressure therapy on a wound that will not stick to the wound.

It is another object of the present invention to provide a medicated foam structure to be used with negative pressure therapy on a wound that will contain natural or synthetic compounds that may promote wound healing.

And yet another object of the present invention is to provide a flexible cover over the wound.

Another object of the present invention is to provide a system involving the application of negative pressure onto a wound in a contained area with a flexible cover over the wound.

Other objects and advantages will become apparent upon reading the specification of the appended claims.

### BRIEF DESCRIPTION OF THE DRAWINGS

The present invention will be described with reference to the attached drawings which are given as non-limiting examples only in which

FIG. 1 depicts a top view of a foam pad loaded with a therapeutic agent or medications.

FIG. 2 depicts a cross-section of the medicated foam pad taken along lines 2-2 of FIG. 1;

FIG. 3 depicts the operation of the foam pad wherein the medication is shown passing through the foam pad unto the skin or open wound of the patient;

FIG. 4 depicts a flexible bag structure for fitting over a limb dressed with the foam pad;

FIG. 5 depicts a flexible bag structure including a leg and foam structure as represented by broken lines inside the bag;

FIG. 6 is an enlarged cross-sectional view of the flexible structure showing a dimpled surface structure;

FIG. 7 depicts an enlarged cross-sectional view of two walls of the flexible structure showing the meeting of its dimples and areas for drawing liquid;

FIG. 8 depicts a side view of a flexible piece or membrane with its medicated open cell face foam structure attached to one side, ready to be applied to the surface of the skin of a patient;

FIG. 9 depicts a cross-sectional view of a flexible piece or membrane with dimples on one side covered with the medicated open cell face foam structure.

FIG. 10 depicts a medicated foam structure in the shape of a hand wherein the therapeutic agent is distributed inside the foam structure; and

FIG. 11 depicts a flexible glove, that is placed securely over the hand shaped foam structure of FIG. 10, so that V.A.C. or negative pressure can be applied to the foam structure.

### DETAILED DESCRIPTION OF THE PREFERRED EMBODIMENT

The present invention may be embodied in either specific forms without departing from the spirit or essential attributes thereof, and it is therefore desired that the present embodiment must be considered in all aspects illustrative and not restrictive, reference being made to the appendant claims rather than to the foregoing description to indicate the scope of the invention.

The present invention generally comprises a wound dressing for cleaning, debriding and treating wounds and open sores. The invention will be described with reference to negative pressure wound therapy or vacuum assisted closure. Patients can benefit from vacuum assisted drainage for removal of debris and infectious material. The medicated gel foam can be used separately from negative pressure wound therapy for cleaning, disinfecting and treating wounds. Its description relating to use with negative pressure therapy or vacuum assisted closure system is not intended to be limitative. Wounds on any area or part of the body can be treated, for example on a digit, limb, part of a limb, hand, leg, etc. The wound may be a chronic open wound, diabetic ulcer, acute wounds, burns, flaps, traumatic ulcers, skin grafts, chronic sub acute wounds, pressure ulcers, diabetic foot ulcers, dehisced wounds and the like. Indeed, any patient would benefit from negative pressure therapy or vacuum assisted drainage and removal of infections would benefit from the present invention.

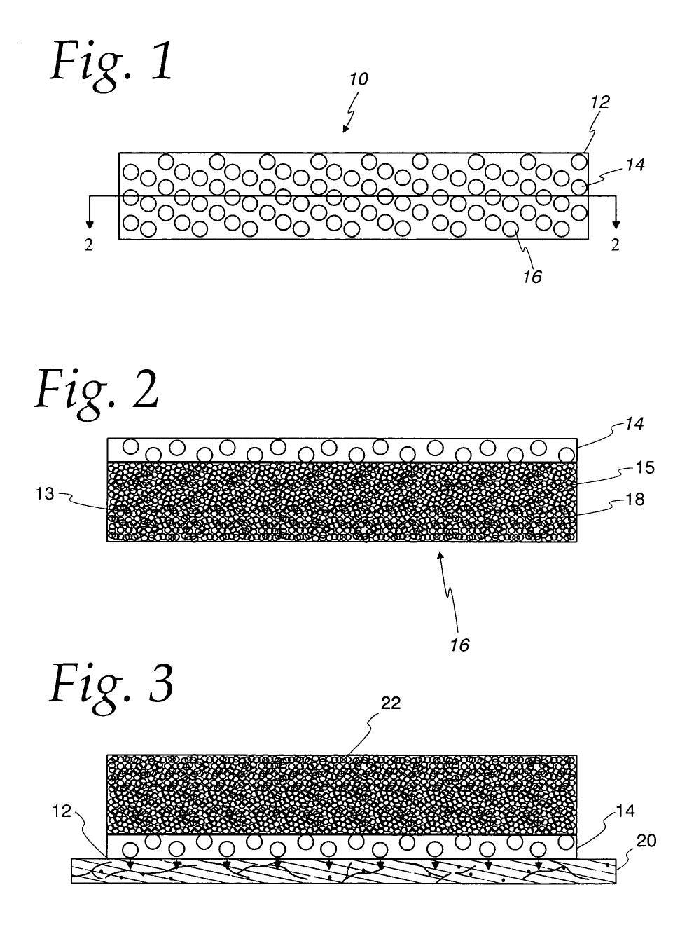

Referring to the drawings, FIG. 1 illustrates the open face foam structure 10 coated on its first side 12 with a therapeutic agent 14, which is preferably silicone gel, with the structure having at least a second side 16. It may be shaped in any desirable configuration, for it is preferably cut by scissors or the like to a sufficient size to cover the wound, not shown in the drawings, to be treated. It may contain edges 13 and 15 that are defined by the first and second sides. The silicone gel may be any medical grade silicone gel. It has non-stick properties. It may be a therapeutic agent and carrier for other therapeutic agents and ingredients. Other therapeutic agents that can be applied to the foam structure for subsequent contact and absorption into the skin include collagenases, antibacterials, antivirals, antifungals, wound modulating factors, for example, growth factors such as platelet derived growth factors, or a protease modulating matrix, natural substances, for example, vitamin A, vitamin C, vitamin E, tea tree oil, aloe vera and emu oil. Additional ingredients or therapeutic agents can be added to the medical grade silicone gel, including but not limited to antioxidants, vitamins, antibacterials, antibiotics, antiseptics, wound healing or growth factors, wound environment modulating factors, growth enhancement factors and other materials or drugs. The additives are incorporated by conventional procedures, for example, blending, layering, etc. into or on the silicone gel. These therapeutic agents can be safely used with, and compliment negative pressure therapy or the V.A.C. system. The negative pressure or vacuum is drawn through the foam structure that entraps tissue, fluids, dried blood, etc. The silicone gel does not interfere with this process and is applied in a fashion that does not interfere with the open-cell structure of the foam. Hence, when negative pressure is applied to one cell of the foam it is also applied to all other cells in the foam. The silicone gel remains in place on the foam structure. The additives leach out of the silicone gel providing wound-healing effects to the site. Due to the non-stick properties of the silicone gel, the foam will not become adherent to the wound, facilitating dressing changes and minimizing wound trauma during extraction of the foam from the wound.

FIG. 2 is a cross section taken at lines 2-2 of the open cell face foam structure of FIG. 1 showing the second side 16 and open face foam structure 18, as well as the therapeutic agent 14. The open face cells of the foam structure typically have diameters sufficient for holding and releasing the therapeutic agent to allow body fluids and tissue pass therethrough under negative pressure. Typically, the diameter may be from about 500 to about 700 microns.

FIG. 3 shows the first side 12 with therapeutic agent 14 contacting skin 20. The therapeutic agent migrates or leaches out of the silicone gel onto the surface of the skin for absorption. The preferred therapeutic agent, which is also silicone gel, with its non-stick properties has been demonstrated to be advantageous in a variety of uses to prevent damage to healing wounds and facilitate would healing. Indeed, the product of the present invention is suitable for the treatment of open wounds when adhesion to the base of the wound is of a concern. This usage includes, but is not limited to any open wound, skin grafts, wounds involving or adjacent to mucosal surfaces, wounds involving internal organs such as the gastrointestinal tract, heart or lungs, venous ulcers, arterial ulcers, diabetic or neurotrophic ulcers, cranial wounds, wounds over blood vessels or nerves, and burns.

The preferred therapeutic agent, silicone gel, can be applied to the open cell foam structure in any conventional manner, for example, the silicone gel can be sprayed on the surface of the foam, pouring the uncured silicone gel onto the foam, applying multiple stripes or designs onto the foam, silk screening the silicone gel onto the foam or dipping the foam into the silicone gel. Excess silicone gel may be removed if necessary by the injection of air into the foam. The silicone gel is typically applied to one side of the foam structure but can also be applied to all surfaces. This may include the inside of a tubular, cylindrical, box-shaped or glove-shaped enclosure.

The flexible porous foam may be a material that is suitable for holding and releasing the therapeutic agent and other additives while allowing the collection and passage therethrough of body fluids and tissues from wound sites when subjected to negative pressure or vacuum. The foam material may be selected from the group consisting of polyurethane, polyvinylacetate, butyl, polyvinyl alcohol, and polyethylene. Preferably, PVA (polyvinyl alcohol) is utilized. It is available from the following companies: Hydrofera LLC Willimantic, Connecticut/USA., Lendell LMI, St. Charles, Mich./USA., Rynel, Boothbay, Me./USA., and Monajackie Int'l Corp. Taipei/Taiwan. The amount or thickness of the foam structure is dependent on its use alone or with negative pressure therapy or vacuum assisted closure. The thickness can be any dimension and as described above, can be fashioned as an “enclosure” for the insertion of a finger, hand, foot or entire limb.

Typically, the foam structure of the present invention is used in treating wounds in negative pressure therapy or vacuum assisted closure as previously described. The foam with its open face cell structure is placed on the wound with the therapeutic agent contacting the wound. A tube is placed in contact with the foam. The foam and wound are covered with an occlusive dressing. The occlusive membrane is applied to secure the skin around the wound forming an airtight seal. The tube is attached to a vacuum and reservoir for disposal. Vacuum or negative pressure is applied to facilitate removal of fluids and tissues from the wound. The vacuum or negative pressure is applied for an appropriate amount of time to cleanse and treat the wound. The use of the therapeutic agent follows its indication, for example, silicone gel provides a non-stick environment and functions as a carrier for other agents. A collagenase would be used in the initial phases to cleanse or debride a wound. Wound environment modulating agents can be used as indicated for the specific product for example platelet-derived growth factor would be used in a diabetic ulcer, aloe vera in a burn, tea, tree oil as an anti-infective agent in burns or contaminated wounds. When bacterial contamination is a concern, antibacterials can be used.

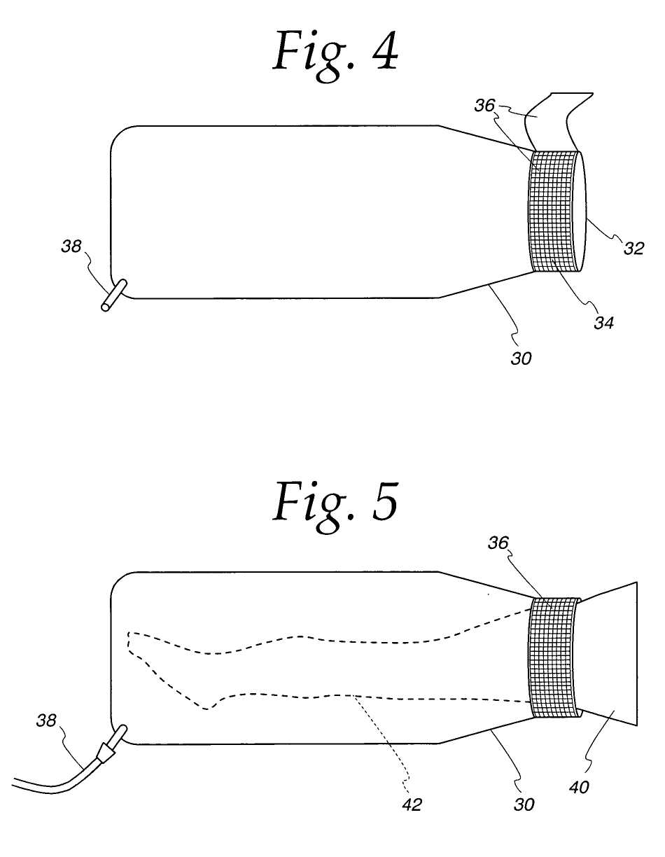

FIG. 4 illustrates an enclosure or bag body 30 for enclosing a body, a limb, partial limb, digit, hand or leg, or other part of the body. Preferably a leg or arm is utilized in this bag. The bag 30 has an adjustable sealable opening 32 with an adhesive 34 in the opening that is compatible with the skin, such as a sticky silicone or hydrogel for securing the device to the body. Hook and loop fastener means 36 can be connected to provide adjustable and comfortable fitting of the bag on a patient and to provide a seal to maintain negative pressure. Outlet drain 38 provides for the passage of fluid therethrough from a leg or limb placed inside the bag 30. Means for applying negative pressure can be attached to drain 38.

FIG. 5 illustrates the use of bag body 30 when a leg 40 is inserted therein. The sealable opening 32 is closed around the leg by hook and loop fastener means 36. Medicated foam structure 42 is shown wrapped around leg 40 inside the bag. The medicated foam structure with its open cell structure is coated with silicone gel, contacting the leg.

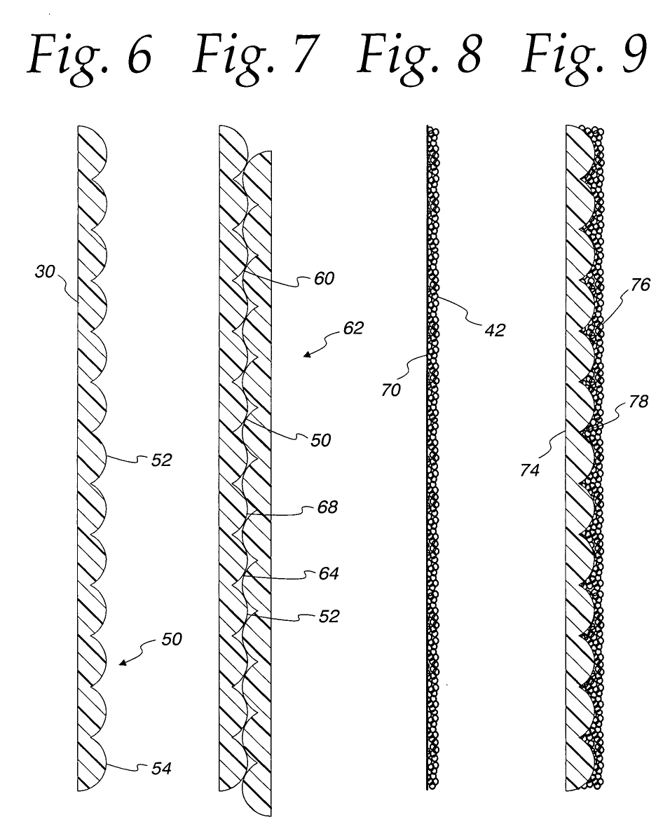

Referring to FIG. 6, therein is a magnified cross-sectional view of the inside 50 of bag body 30 showing an inner surface 52 having dimpled or ridged surface 54.

FIG. 7 shows two inner surfaces 50 and 60 from the walls of the bag meeting each other in a collapsed position 62 showing two dimpled surfaces contacting each other. This contact occurs when the bag 30 collapses under suction which is applied to the bag through outlet drain 38. The suction is sufficient so that the two inner surfaces 50 and 60 contact each other. The dimpled surfaces 52 and 64 when contacting each other form voids 68 between the dimples. Negative pressure applied to outlet drain 38 will extract fluids located in the voids 68.

A flat flexible piece or membrane 70 made from any suitable material, preferably the same as bag body 30 with the medicated porous open cell foam structure 42 attached to one side is shown in FIG. 8. This versatile flexible piece can be used almost anywhere on the body to cover a wound. A drain can be inserted in any convenient location for the application of negative pressure or a vacuum to drain liquid from the wound. The flat flexible piece can be attached to the body by adhesive.

FIG. 11 depicts a flexible sheet 74 with dimples 78 on one side. Medicated porous open cell foam structure 76 is attached over dimples 78. The flexible dimpled sheet can be attached to the skin of the patient by an adhesive. A drain can be inserted at any convenient location of the piece for the application of negative pressure or vacuum to drain liquids from the wound.

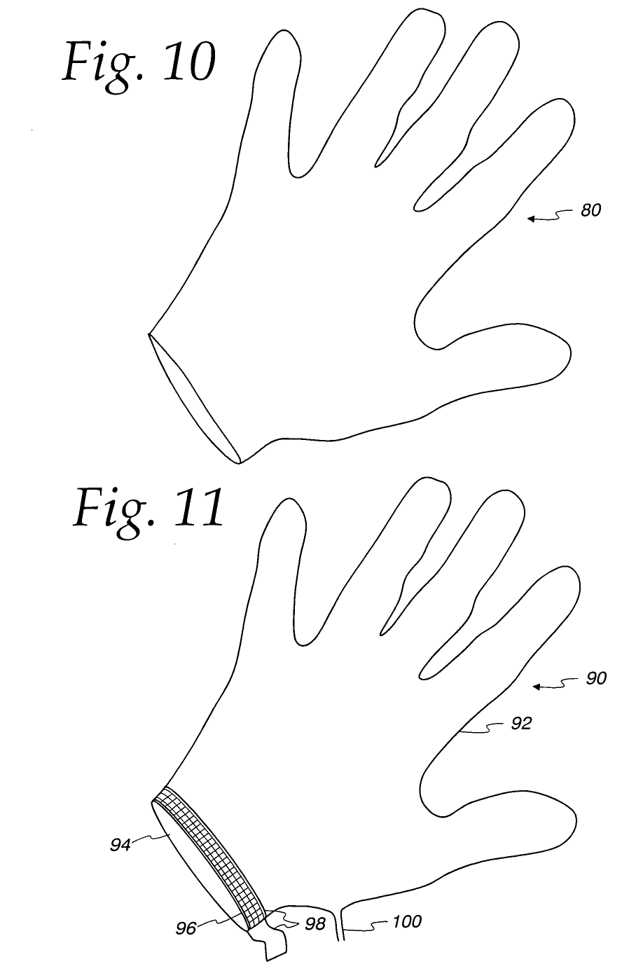

A medicated foam structure in the shape of a hand or glove 80 is depicted in FIG. 10. The glove 80 fits securely on the hand of a patient for treating wounds on the entire hand or portion thereof. Open foam cells hold non-sticky silicone gel and/or other therapeutic agent.

Body bag 90 is shown in FIG. 11 in the shape of a glove 92 for securely positioning over medicated foam glove 80. The shape of the bag 90 can alternatively be for a limb, digit, leg or foot for it is immaterial to the functioning of the medicated foam structure. The different shapes provide convenient alternatives to the physician in the treatment of the patient. Sealable opening 94 with adhesive 96 and hook and loop connectors 98 adjustably secure the glove 92 to the wrist of the patient for the application of negative pressure. Drainage outlet 100 provides for passage of fluids from the wounds and provides for the application of negative pressure.

Although this invention has been described with reference to a preferred embodiment, obvious modifications and alterations of the invention may be made without departing from the spirit and scope of the invention. The preferred application of the present invention is for the treatment of wounds with negative pressure therapy or vacuum assisted closure of wounds, however, the invention can be utilized to treat wounds without using the medicated gel foam structure without the aforementioned treatments.

1. A medicated foam structure comprising:
a foam structure having first and second sides, and
a therapeutic agent applied on at least one side.

2. The medicated foam structure according to claim 1 wherein the structure of the foam comprises porous open cells with a diameter of about 500 microns to about 700 microns.

3. The medicated foam structure according to claim 1 having wherein the structure has side edges.

4. The medicated foam structure according to claim 1 wherein the therapeutic agent is selected from the group consisting of silicone gel, hydrogels, collagenases, antibacterials, antivirals, antifungals, wound modulating factors, growth factors such as platelet derived growth factors, protease modulating matrix, natural substances, vitamin A, vitamin C, vitamin E, tea tree oil, aloe vera and emu oil.

5. The medicated foam structure according to claim 1 wherein the therapeutic agent is applied to one or more sides of the structure so the open cell can hold and subsequently release the therapeutic agent.

6. The medicated foam structure according to claim 1 wherein the structure holds sufficient quantities of therapeutic agents to treat wounds and not to obstruct the porous open cell structure.

7. The medicated foam structure according to claim 1 wherein the therapeutic agent is applied to the one side to form a layer of therapeutic agent with the open cells of the structure in a thickness of about 150 microns to about 500 microns.

8. The medicated foam structure according to claim 1 wherein the therapeutic agent is applied to the structure by methods selected from the group consisting of spraying, dipping the foam into a liquid therapeutic agent, and pouring the liquid therapeutic agent onto the foam structure.

9. The medicated foam structure according to claim 1 wherein it is shaped as a limb, glove, leg or foot.

10. A method of treating wounds by applying a medicated foam structure to the wound, the structure having at least first and second sides and a therapeutic agent applied to at least one side.

11. The method according to claim 10 wherein the structure of the foam comprises porous open cells with a diameter of about 500 to about 700 microns.

12. The method according to claim 10 wherein the therapeutic agent is selected from the group consisting of silicone gel, hydrogels, collagenases, antibacterials, antivirals, antifungals, wound modulating factors, a protease modulating matrix and natural substances.

13. The method according to claim 12 where its wound modulating factor is a growth factor.

14. The method according to claim 12 where natural substance is released from the group consisting of Vitamin A, Vitamin C, Vitamin E, tea tree oil, aloe vera and emu oil.

15. The method according to claim 10 wherein the therapeutic agent is applied to at least the first side so the open cell holds and subsequently releases the therapeutic agent.

16. The method according to claim 10 wherein the structure holds sufficient quantities of therapeutic agents to treat wounds and not to obstruct the porous open cell structure.

17. The method according to claim 10 wherein the therapeutic agent is applied to at least the first side to form a layer of therapeutic agent with the open cells of the structure in a thickness of about 150 microns to about 500 microns.

18. The method according to claim 10 wherein the therapeutic agent is applied to either or both sides by methods selected from the group consisting of spraying, dipping the foam into a liquid therapeutic agent, and pouring the liquid therapeutic agent onto the foam structure.

19. The method according to claim 10 wherein negative pressure is applied to the wound through the medicated foam structure to promote healing.

20. The method according to claim 10 when the wound to be treated is selected from the group consisting of chronic open wounds, diabetic ulcers, acute wounds, traumatic wounds, skin grafts, chronic subacute wounds, burns, flaps, traumatic ulcers, pressure ulcers, and dehisced wounds.

21. A medicated foam structure comprising a foam structure having first and second sides with the second side defining an enclosure for accepting parts of the body to be treated selected from the group consisting of a limb, digit, hand, foot or part of a limb wherein a therapeutic agent is applied to the second side which is in contact with the part of the body to be treated with the therapeutic agent.

22. The medicated foam structure according to claim 21 wherein the structure of the foam comprises porous open cells with a diameter of about 500 microns to about 700 microns.

23. The medicated foam structure according to claim 21 wherein the therapeutic agent is selected from the group consisting of silicone gel, hydrogels, collagenases, antibacterials, antivirals, antifungals, wound modulating factors, growth factors, platelet derived growth factors, a protease modulating matrix, natural substances, vitamin A, vitamin C, vitamin E, tea tree oil, aloe vera and emu oil.

24. The medicated foam structure according to claim 21 wherein the therapeutic agent is applied to one or more sides of the structure so the open cell can hold and subsequently release the therapeutic agent.

25. A system for treating a wound comprising:
a. a medicated foam structure having first and second sides, and a therapeutic agent applied on at least one side that is applied to the wound,
b. a flexible bag body enclosing the wound and medicated foam structure, said body bag having an adjustable sealable opening and
c. means for applying negative pressure to the wound through the medicated foam structure to promote healing.

26. The medicated foam structure according to claim 25 wherein the structure of the foam comprises porous open cells with a diameter of about 500 microns to about 700 microns.

27. The medicated foam structure according to claim 25 wherein the structure has side edges.

28. The medicated foam structure according to claim 25 wherein the therapeutic agent is selected from the group consisting of silicone gel, hydrogels, collagenases, antibacterials, antivirals, antifungals, wound modulating factors, growth factors, platelet derived growth factors, a protease modulating matrix, natural substances, vitamin A, vitamin C, vitamin E, tea tree oil, aloe vera and emu oil.

29. The medicated foam structure according to claim 25 wherein the therapeutic agent is applied to one or more sides of the structure so the open cell can hold and subsequently release the therapeutic agent.

30. The medicated foam structure according to claim 25 wherein the structure holds sufficient quantities of therapeutic agents to treat wounds and not to obstruct the porous open cell structure.

31. The system according to claim 25 wherein the flexible bag has an adjusted opening with sealing means.

32. The system according to claim 31 wherein the sealing means comprises hook and loop connectors fasteners.

33. The system according to claim 25 wherein the flexible bag has at least one outlet for draining fluids.

34. The system according to claim 25 wherein the flexible bag has means for applying negative pressure to the wound through the medicated foam structure to promote healing.

35. The system according to claim 25 wherein the flexible bag has walls and an inside surface having a dimpled surface such that when the bag collapses from the application of negative pressure onto itself, voids are formed between the walls forming draining pathways for any entrapped fluids draining from the wound due to the application of negative pressure.

36. The system according to claim 25 wherein the medicated foam structure is in the shape of a glove.

37. The system according to claim 36 wherein the flexible body bag is in the shape of a glove.

38. The system according to claim 25 wherein a flexible sheet is utilized in place of the flexible body bag for covering a wound.

39. The system according to claim 30 wherein a dimpled flexible sheet is utilized in place of the flexible body bag for covering a wound.
